(19) United States
(12) Patent Application Publication
(10) Pub. No.: US 2017/0035275 A1
(43) Pub. Date: Feb. 9, 2017

(54) CONNECTION ADAPTER FOR OPTICAL FIBER AND ENDOSCOPE DEVICE

(71) Applicant: OLYMPUS CORPORATION, Tokyo (JP)

(72) Inventors: Hiroyoshi YAJIMA, Tokyo (JP); Mitsuru NAMIKI, Tokyo (JP)

(73) Assignee: OLYMPUS CORPORATION, Tokyo (JP)

(21) Appl. No.: 15/297,510

(22) Filed: Oct. 19, 2016

Related U.S. Application Data
(63) Continuation of application No. PCT/JP2015/002104, filed on Apr. 16, 2015.

(30) Foreign Application Priority Data
Apr. 21, 2014 (JP) 2014-087513

(51) Int. Cl. A61B 1/00; G02B 6/38; G02B 23/26; A61B 1/07

(52) U.S. Cl. CPC A61B 1/00126; A61B 1/07; G02B 6/3825; G02B 6/3877; G02B 6/3871; G02B 6/3843; G02B 23/26

(57) ABSTRACT

A connection adapter includes: an adapter housing; a split sleeve; and a spacer having a space determiner disposed inside the split sleeve and an angle determiner that passes through the slit of the split sleeve to be secured relative to the adapter housing. The two connector connection pars are each connected to the connectors, respectively, causing the ferrules of the connectors to be fitted into the split sleeve and secured across the spacer, so that the single mode optical fibers of the connectors are optically connected to each other via no point of contact therebetween, to thereby provide a connection adapter capable of creating stable connection efficiency for optical fibers and an endoscope device using the connection adapter.

### CROSS REFERENCE TO RELATED APPLICATIONS

The present application is a Continuing Application based on International Application PCT/JP2015/002104 filed on Apr. 16, 2015, which, in turn, claims priority from Japanese Patent Application No. 2014-87513 filed on Apr. 21, 2014, the entire disclosure of these earlier applications being incorporated herein by reference.

### TECHNICAL FIELD

Disclosed is a connection adapter for an optical fiber and an endoscope apparatus using the connection adapter.

### BACKGROUND

Optical fiber connection adapters are used for connecting an optical fiber disposed outside a casing to the inside of the casing which is connected to a laser light source or has a laser light source incorporated therein. The optical fiber disposed outside the casing may desirably be configured to be readily attached to/detached from the casing, for the purpose of maintenance of the apparatus or reconfiguration of the components.

For example, the field of endoscope apparatus has seen developments in recent years in endoscopes including, for example, a laser scanning endoscope, a confocal endoscope, and an endoscope equipped with a laser light source, in which: the laser scanning microscope vibratorily drives a leading end of the scope in a body cavity of a specimen to scan and irradiate the inspection site with laser light and detects resulting reflected light, to thereby generate a two-dimensional image; the confocal endoscope employs confocal technology to obtain a clear image that is high in magnification as well as definition; and the endoscope equipped with a laser light source uses the light source to generate white light by a fluorescent material to illuminate the inspection sight therewith. These apparatuses use a single mode optical fiber to transmit illumination light from the laser light source to the scope leading end.

In general, endoscope apparatuses for use in biological observation are configured to insert the scope partially into a body cavity. Thus, the apparatuses are structured to have the scope detachable from the endoscope body with light sources or the like incorporated therein, for the sake of cleaning operation after use. A conventional endoscope apparatus using lamp illumination includes a lamp disposed in a casing of the endoscope body, and guides light of the lamp to the leading end of the scope through, for example, light guide bundles of light guides each having a diameter of less than 100 μm. Optical fiber connection technology used in optical fiber communication is employed to have the light guide bundles abutted to each other between the endoscope body and the scope, to thereby transmit light therethrough.

According to the optical fiber connection technology used in optical communication, an optical adapter having a split sleeve is used to connect optical fiber connectors each having a ferrule which incorporates therein a leading end of an optical fiber. The ferrule of each of the optical fibers to be connected is inserted into the split sleeve from both sides of the optical fiber adapter such that the cores of the optical fibers are abutted to each other in the split sleeve. The split sleeve is formed of a hard material such as zirconia, and holds the ferrules aligned with each other. Further, as suggested in, for example, JP2005300594A (hereinafter Patent Literature (PTL) 1), the split sleeve may accommodate therein a gradient index (GRIN) lens such as to connect the ferrules to each other via the gradient index (GRIN) lens. The use of GRIN lens allows for enlarging the mode field diameter of the optical fibers, to thereby enhance connection efficiency.

### CITATION LIST

### Patent Literature

PTL 1: JP2005300594A

### SUMMARY

On the other hand, however, when abutting leading ends of optical fibers to each other or abutting an optical fiber to a GRIN lens for connection, the optical fibers may collect dust on the end faces thereof, which may break the optical fibers when abutted, causing a fatal error. To prevent such an error, a cleaning operation is needed each time the scope is connected to the endoscope body, which will impair the convenience of the user of the endoscope apparatus. In light thereof, we have made extensive studies for accommodating GRIN lenses in the tips of ferrules of both optical fiber connecters to be connected to thereby leave a gap between the GRIN lenses when the connectors are connected to an adapter. When the connectors are connected via GRIN lenses, laser light travelling through a single mode optical fiber can be expanded in spot diameter, which allows for increasing connection efficiency.

However, in an endoscope using a single mode optical fiber for visible light, the optical fiber will have an extremely smaller core diameter. For example, an endoscope using laser light has a core diameter of about 3.5 μm, which is much smaller than the core diameter of about 10 μm of an optical fiber for optical communication using near-infrared light. Here, the use of GRIN lens may expand the spot diameter of laser light; however, the non-contact space between the GRIN lenses may slightly vary or the split sleeve may rotate inside the optical fiber to change its angle each time the optical fibers are connected, which varies the connection efficiency of the optical fibers.

It could be helpful to provide a connection adapter connecting connectors to each other, the connectors each having a ferrule which incorporates therein a leading end of a single mode optical fiber, the connection adapter including:
an adapter housing having two connector connection parts opposing to each other;
a split sleeve disposed between the two connector connection parts; and
a spacer having a space determiner disposed in the split sleeve and an angle determiner that passes through the slit of the split sleeve to be secured relative to the adapter housing,
in which the two connector connection parts are each connected to each of the connectors, causing the ferrules of the connectors to be fitted into the split sleeve and secured across the spacer, so that the single mode optical fibers of the connectors are optically connected to each other via no point of contact therebetween.

Preferably, the angle determiner may be configured to be capable of adjusting angles around the central axis of the split sleeve with respect to the adapter housing.

The space determiner may include a plurality of plate-like members, and when one of the connector connection parts is not connected to the connector, at least some of the plurality of plate-like members may be tilted at different angles toward the connector connection part side not connected to the connector, so as to shield an optical path of light emitted from the single mode optical fiber of the connector connected to the other connector connection part.

Further, the adapter may further include an optical detection unit between the adapter housing and the split sleeve. In this case, the optical detection unit may be disposed on each side of the two connector connection parts of the spacer.

It could also helpful to provide an endoscope apparatus, including:
a casing which accommodates therein a laser light source or is connected to a laser light source;
a scope which irradiates an object with laser light output from the casing and receives signal light obtained from the object;
an image processor which generates an image, based on the signal light received by the scope; and
a connection adapter which connects, between the casing and the scope, connectors each having a ferrule which incorporates therein a leading end of an optical fiber,
in which the connection adapter includes:
   an adapter housing having two connector connection parts opposing to each other;
   a split sleeve disposed between the two connection parts; and
   a spacer having a space determiner disposed in the split sleeve and an angle determiner that passes through the slit of the split sleeve to be secured to the adapter housing,
in which the two connector connection parts are each connected to each of the connectors, causing the ferrules of the connectors to be fitted into the split sleeve and secured across the spacer, so that the single mode optical fibers of the connectors are optically connected to each other via no point of contact therebetween.

### BRIEF DESCRIPTION OF THE DRAWINGS

In the accompanying drawings:

FIG. 1 is a top view of the disclosed adapter and connectors according to Embodiment 1;

FIG. 2 is a longitudinal section of the adapter and the connectors of FIG. 1;

FIG. 3 is a longitudinal section of the adapter and the connectors of FIG. 1 which are coupled to each other;

FIG. 4A is a schematic illustration of a longitudinal section of a connection part of single mode optical fibers;

FIG. 4B is a schematic illustration of a section taken along the line A-A′ of FIG. 4A;

FIG. 5 is a longitudinal section of the adapter according to Modified Example 1;

FIG. 6A is a schematic illustration of a longitudinal section of a connection part of single mode optical fibers according to Modified Example 2;

FIG. 6B is a schematic illustration of a section taken along the line A-A′ of FIG. 6A;

FIG. 7 is a longitudinal section schematically illustrating a connection part of single mode optical fiber, which uses the disclosed adapter according to Embodiment 2;

FIG. 8 is a longitudinal section schematically illustrating the connection part of single mode optical fibers, which uses the disclosed adapter according to Embodiment 3;

FIG. 9 is a schematic external view of an endoscope apparatus with the disclosed adapter incorporated therein; and

FIG. 10 is a block diagram illustrating a schematic configuration of the endoscope apparatus of FIG. 9.

### DETAILED DESCRIPTION

Hereinafter, Embodiments of the disclosed adapter and apparatus will be illustrated with reference to the drawings.

### Embodiment 1

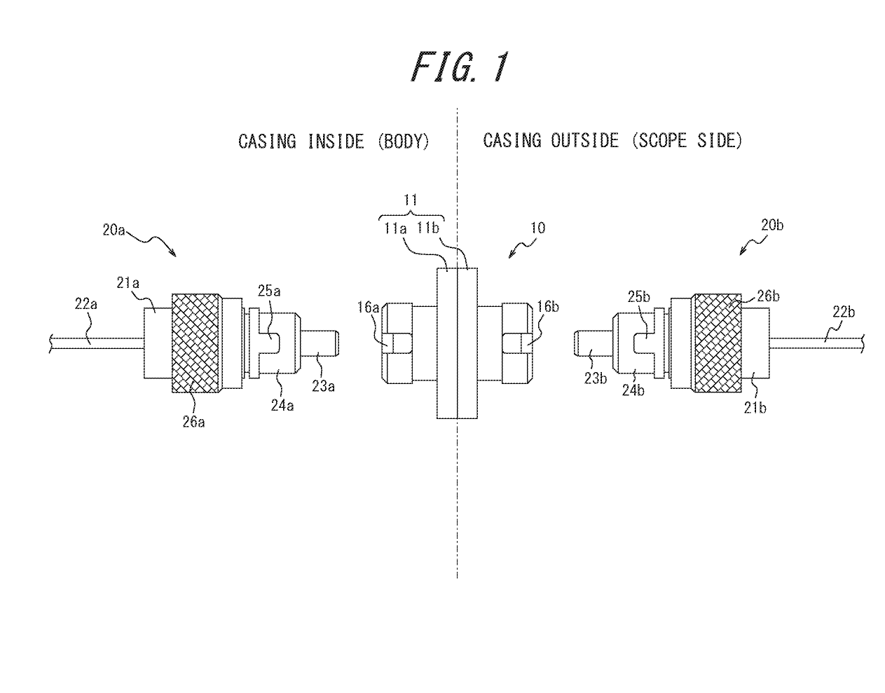

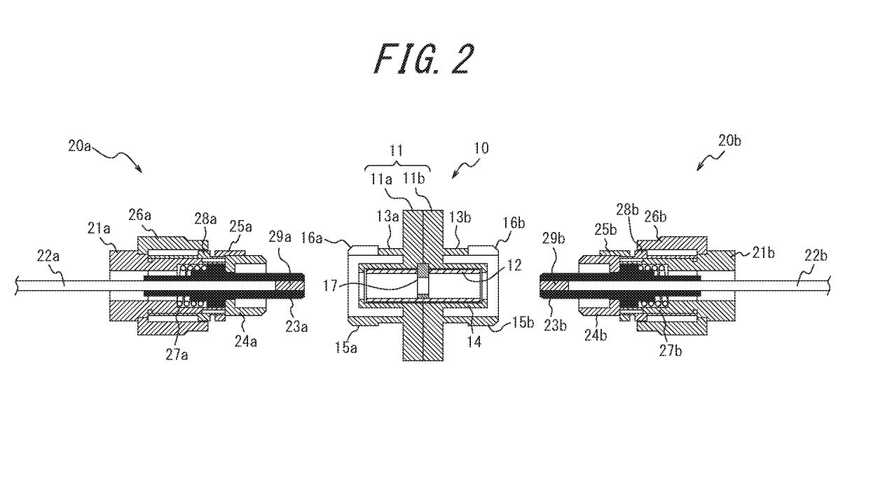

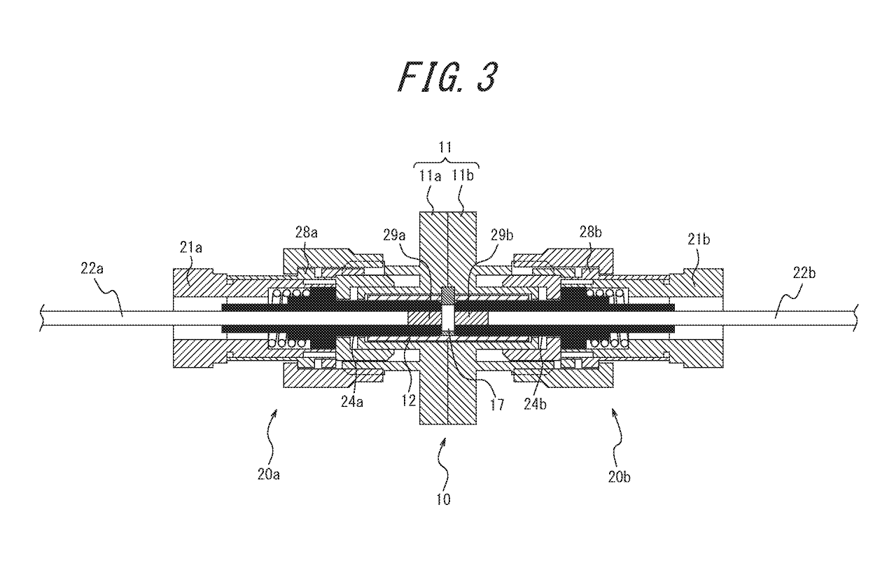

The disclosed adapter and connector according to Embodiment 1 are described with reference to FIGS. 1 to 3. FIG. 1 is a top view of an adapter 10 and connectors 20a, 20b according to Embodiment 1. FIG. 2 is a longitudinal section of the adapter 10 and the connectors 20a, 20b of FIG. 1. FIG. 3 is a longitudinal section of the adapter 10 and the connectors 20a, 20b of FIG. 1 which are coupled to each other.

The adapter 10, which is an optical fiber connection adapter, connects the connectors 20a, 20b to each other, between the inside of a casing accommodating a laser light source or a casing connected to a laser light source and the outside of the casing, the connectors 20a, 20b having ferrules 23a, 23b which incorporates therein leading ends of single mode optical fibers 22a, 22b, respectively. The adapter 10 is arranged on a face of the casing, and connects the connector 20a disposed inside the casing to the connector 20b disposed outside the casing.

As illustrated in FIG. 2, the adapter 10 includes an adapter housing 11 and a split sleeve 12. The adapter housing 11 is formed of two members 11a and 11b which are coupled to each other and each disposed on the casing inside and the casing outside, respectively. The adapter housing 11 has an outer cylindrical part 13a having an opening on the casing inside and an outer cylindrical part 13b having an opening on the casing outside. The outer cylindrical parts 13a, 13b has, on the inside thereof, an inner cylindrical part 14 having a cavity, between the connector 20a side and the connector 20b side. The split sleeve 12 in a cylindrical shape is disposed inside the cavity of the inner cylindrical part 14. The inner cylindrical part 14 has inner peripheral surfaces on both sides protruding inward to prevent the split sleeve 12 from being detached. Further, the outer cylindrical parts 13a, 13b have external threads (male threads) 15a, 15b disposed on the outer peripheral end sides. Further, grooved key receivers 16a, 16b are disposed in part of the inner peripheral surface of the outer cylindrical parts 13a, 13b. As described above, the adapter housing 11 has, on the casing inside and the casing outside, two mutually-opposing connector connection parts each having a shape connectable to the connectors 20a, 20b, respectively. The outer cylindrical parts 13a, 13b, the inner cylindrical parts 14 and the external threads 15a and 15b are the connector connection parts.

The split sleeve 12 is a hollow tubular member having a slit extending in a longitudinal direction (direction along the central axis when disposed inside the inner cylindrical part 14), and is formed of hard ceramics such as zirconia. A spacer 17 is disposed at the longitudinal direction center of the split sleeve 12. The spacer 17 is disposed inside the split sleeve 12 and abutted by the ferrules 23a, 23b of the connectors 20a, 20b, to thereby define a space between the ferrules 23a, 23b, while part of the spacer 17 passes through the slit of the split sleeve 12 so as to protrude from the split sleeve 12 to be fitted into the adapter housing 11 and secured.

The connector 20a is configured by including a connector housing 21a and a ferrule 23a which incorporates therein a leading end of the single mode optical fiber 22a. Hereinafter, the leading direction of the single mode optical fiber 22a of the connector 20a is referred to as forward while the direction opposite thereto is referred to as backward.

A tip part of the connector housing 21a is formed as a cylindrical part 24a having a cylindrical wall part, which is in a shape to be fitted into a gap between the inner cylindrical part 14 and the outer cylindrical part 13a of the adapter 10. Further, a key 25a is protrudingly formed on the outer peripheral surface of the cylindrical part 24a. The key 25a fits into the key receiver 16a of the adapter 10 to be engaged therewith when the adapter 10 and the connector 20a are coupled to each other, to thereby accurately align the adapter 10 and the connector 20a in the rotation direction.

On the outer periphery of the connector housing 21a, a coupling nut 26a is disposed as being rotatable and movable in the fiber optical axis direction within a specific range. An internal thread (female thread) is disposed on the inner surface of the coupling nut 26a, which is configured to mesh with the external thread 15a of the outer cylindrical part 13a of the adapter housing 11.

The ferrule 23a is in a columnar shape chamfered at the tip end, and has the single mode optical fiber 22a inserted therethrough along the central axis thereof. The columnar part of the ferrule 23a protrudes forward from the center of the cylindrical part 24a of the connector housing 21a, and has the outer periphery supported by the connector housing 21a, behind the cylindrical part 24a. Further, the continuing rear side of the ferrule 23 is provided with a flange, which is slidable relative to the inner peripheral surface of the adapter housing 11 in the optical axis direction of the single mode optical fiber 22a in the adapter housing 11 within a specific range and biased forward by a spring 27a disposed inside the adapter housing 11.

A lens 29a is accommodated in a tip end of the ferrule 23a. The lens 29a emits lights having been transmitted through the core of the single mode optical fiber 22a, as parallel lights expanded in spot diameter. Alternatively, the light is emitted as convergent light. As the lens 29a, a gradient index (GRIN) lens having a diameter similar to that of the single mode optical fiber 22a may be used. At this time, the lens 29a and the single mode optical fiber 22a are brought into contact with each other, or fusion spliced through glass materials or in a fixed state with a certain gap therebetween.

In the above, the connector 20a disposed on the casing inside has been described. However, the connector 20b disposed on the casing outside is similarly configured. Here, the connector 20a in the casing basically maintains connection over a long period of time, while the connector 20b outside is attached/removed more often than the connector 20a.

With the aforementioned configuration, when connecting the connectors 20a, 20b to the adapter 10, a tip end of the adapter 10 and tip ends of the connectors 20a, 20b are aligned in position in the rotation direction such that the both axes coincide with each other and the keys 25a, 25b of the connectors 20a, 20b are to be fitted into the key receivers 16a, 16b of the adapter 10, before fitting the ferrules 23a, 23b into the split sleeve 12 and fitting the cylindrical parts 24a, 24b of the connectors 20a, 20b into between the outer cylindrical parts 13a, 13b of the adapter 10 and the both ends of the inner cylindrical part 14.

Next, the coupling nuts 26a, 26b are moved to the adapter 10 side and rotated. This causes the external thread 15a of the adapter housing 11 and the internal thread of the coupling nut 26a to mesh with each other, which moves forward the coupling nuts 26a, 26b toward the adapter 10 side. As a result, the ferrule 23a further slides forward inside the split sleeve 12.

When a tip of the ferrule 23a of the connector 20a on the casing inside and a tip of the ferrule 23b of the connector 20b on the casing outside each abut to the spacer 17, spring force of the springs 27a, 27b in the connectors 20a, 20b presses the ferrules 23a, 23b against the spacer 17 by a pressing force of certain level or less. Steps 28a, 28b formed on the outer periphery of the connector housings 21a, 21b engage with the rotation of the coupling nuts 25a, 26b, to thereby stop the rotation of the coupling nuts 26a, 26b, which prevents excessive pressing force from being generated between the ferrules 23a, 23b.

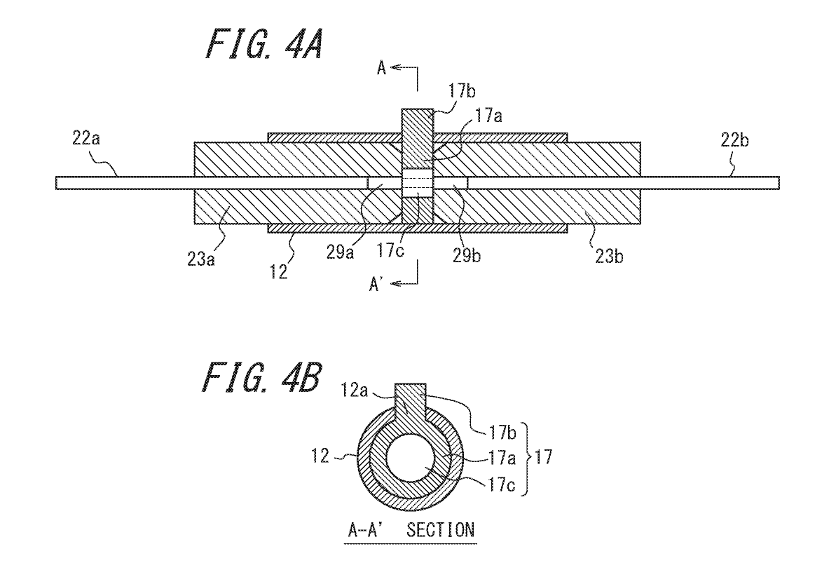

Here, the spacer 17 arranged at the center of the split sleeve 12 is further described. FIG. 4 schematically illustrate the connection part of single mode optical fibers, in which FIG. 4A is a longitudinal section and FIG. 4B is a section taken along the line A-A′ of FIG. 4A. The spacer 17 includes an annular plate-like space determiner 17a along the inner circumference of the split sleeve 12 and an angle determiner 17b protruding from a part of the space determiner 17a to pass through between the slit 12a of the split sleeve 12 and fit into the adapter housing 11.

The space determiner 17a is formed like a flat plate as illustrated in FIG. 4A, and keeps constant the distance between the tip end of the connector 20a and the tip end of the connector 20b when the connectors 20a, 20b are connected to the adapter 10 so that the distance would not vary depending on the connection. With this configuration, the single mode optical fiber 22a and the single mode optical fiber 22b are optically connected to each other with the tip of the lens 29a and the tip of the lens 29b each connected to the fibers 22a, 22b being spaced apart from each other across a stable distance via a gap 17c inside the annular space determiner 17a. Here, the thickness of the spacer 17 or the distance between the connectors 20a and 20b is selected from 0.1 mm to 2 mm, and when the connector 20a emits convergent light, the position of the minimum beam diameter of the convergent light falls within the spacer 17. Further, the minimum beam diameter of the convergent light is larger than the beam diameters of the single mode optical fibers 22a, 22b.

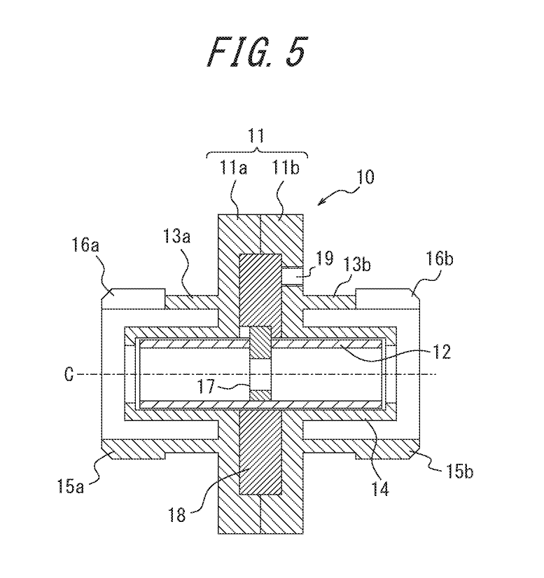

Further, as illustrated in FIG. 4B, when viewed in the central axis direction of the split sleeve 12, i.e., in the optical axis direction of the single mode optical fibers 22a, 22b, the width of the angle determiner 17b is substantially the same as the width of the slit 12a of the split sleeve 12, and the orientation of the angle determiner 17b defines an angle around the central axis C (see FIG. 5) of the split sleeve 12. Accordingly, the angle determiner 17b may be secured with respect to the adapter housing 11, to thereby determine the angle of the split sleeve 12 with respect to the adapter 10.

The rotation of the ferrule 23a is secured with respect to the connector 20a, and the alignment between the adapter 10 and the connector 20a in the rotation direction is secured by the key 25a fitted into the key receiver 16a. Thus, the rotation of the split sleeve 12 may be controlled with respect to the adapter 10, to thereby fix the relation between the rotational angles of the split sleeve 12 and of the ferrule 23a. The same applies to the relation between the adapter 10 and the ferrule 23b of the connector 20b. When the angular relation about the optical axes of the ferrules 23a, 23b and the split sleeve 12 remains the same, variations in connection efficiency may be reduced. This way reduces variations in connection efficiency resulting from the attachment/removal of the connectors 29a, 29b to/from the adapter 10.

As described above, according to Example 1, the spacer 17 is disposed at the center of the split sleeve 12, which allows the lenses 29a, 29b to be connected to each other without via no point of contact therebetween, the space determiner 17a of the spacer 17 stabilizes the distance between the lens 29a, 29b, and the angle of the split sleeve 12 is secured by the angle determiner 17b, which allows the connectors 20a, 20b to be connected to the adapter 10 with stable connection efficiency with less variations for each attachment/removal.

### Modified Example 1

FIG. 5 is a longitudinal section of the adapter according to Modified Example 1 of Embodiment 1. According to Modified Example 1, a spacer holder 18, which is fitted with the angle determiner 17b of the spacer 17, is provided to the outer circumference of the central part of the split sleeve 12. The spacer holder 18 is an annular plate-like member having the split sleeve 12 penetrating through inside thereof, and the angle in the rotation direction is mutually fixed between the split sleeve 12 and the spacer 17. The adapter housing 11 has a disk-like cavity to accommodate the spacer holder 18 therein, and the spacer holder 18 is configured, in the cavity with respect to the adapter housing 11, to be adjustable in rotation about the central axis C of the split sleeve 12. Specifically, the spacer holder 18 has a fixing screw hole (not shown), and the adapter housing 11 has a drilled hole 19 to see therethrough the fixing screw hole despite the rotation of the spacer holder 18. A socket screw is attached to the spacer holder 18 from outside of the adapter housing 11, and the socket screw is used to rotate the spacer holder 18 for adjustment. When the rotational angle is determined, the socket hole is further fastened to fix the relative angle between the adapter housing 11 and the spacer holder 18. The rest of the configuration is similar to that of Embodiment 1.

With the aforementioned configuration, the adapter 10 according to Modified Example 1 increases the degree of freedom in alignment, allowing the angle of the split sleeve 12 to be adjusted via the spacer holder 18 when connecting the connectors 20a, 20b, to thereby obtain high connection efficiency between the single mode optical fibers 22a, 22b.

### Modified Example 2

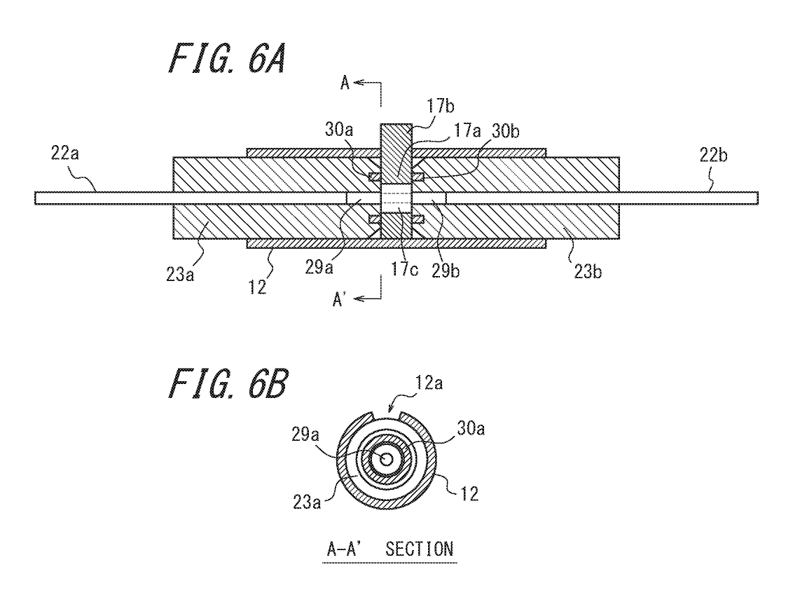

FIG. 6 are schematic illustration of a connection part of single mode optical fibers according to Modified Example 2, in which FIG. 6A is a longitudinal section and FIG. 6B is a section taken along the line A-A′ of FIG. 6A. The ferrules 23a, 23b in Modified Example 2 each have magnets 30a, 30b, respectively, embedded in end faces to be abutted to the spacer 17. The magnets 30a, 30b are annular magnets with the optical axes of the lenses 29a, 29b as centers. However, the shapes of the magnets 30a, 30b are not limited to the annular shape, with various shapes and arrangement available. Meanwhile, the spacer 17 is formed of a magnetic material such as, for example, stainless. The rest of the configuration is similar to that of Embodiment 1. With this configuration, the magnetic forces of the magnets 30a, 30b bring the space determiner 17a of the spacer 17 and the end faces of the ferrules 23a, 23b into intimate contact with each other, when connecting connectors 20a, 20b to the adapter 10. As a result, the space determiner 17a of the spacer 17 and the ferrules 23a, 23b can further reliably be adhered to each other, which can improve reproducibility of connection efficiency regardless of repeated attachment/removal.

### Embodiment 2

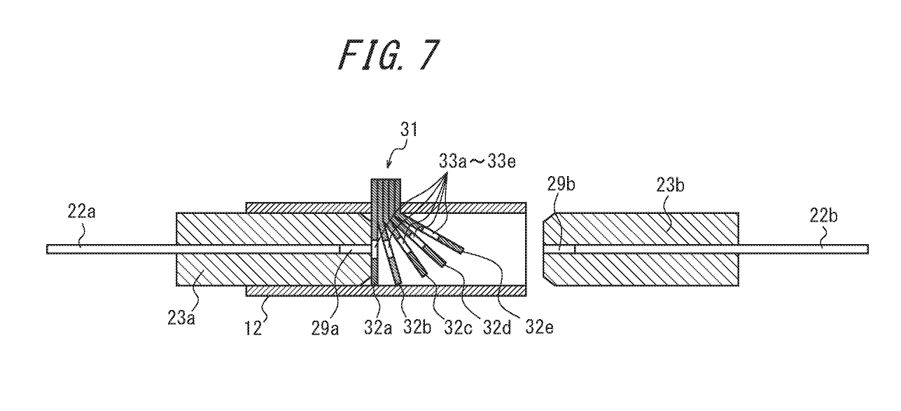

FIG. 7 is a longitudinal section schematically illustrating a single mode optical fiber connection part, which uses the disclosed adapter according to Embodiment 2. The adapter 10 of Embodiment 2 uses a spacer 31 including a plurality of leaf springs 32a to 32e in place of the space determiner 17a of the spacer 17 of Embodiment 1. Of those, the leaf spring 32a is always perpendicular to the central axis of the split sleeve 12. In contrast, when the connector 20b is not connected, i.e., in a state where the ferrule 23b is not fitted into the split sleeve 12, the leaf springs 32b to 32e are each tilted at different angles toward the casing outside (on the connecter connection part side with no connector connected thereto). Here, the leaf springs 32b to 32e are each formed in an annular plate shape having an outer peripheral diameter smaller than an inner peripheral diameter of the split sleeve 12, which allows tilting of the springs inside the split sleeve 12. An optical path of light emitted from the single mode optical fiber 22a via the lens 29a with the leaf springs 31b to 32e being tilted runs into any of the leaf springs 32b to 32e to be shielded.

Meanwhile, in connecting the connector 20b to the adapter 10, the ferrule 23b is inserted from the right (casing outside) of the split sleeve 12 of FIG. 7. When the tip end of the ferrule 23b reaches the leaf spring 32e, the ferrule 23b further advances into the split sleeve 12, so as to sequentially push, by the tip end thereof, and stretch the leaf springs 32e, 32d, 32c, 32b, 32a. The ferrule 23b thus inserted into the split sleeve 12 eventually causes the leaf springs 32a to 32e to be stretched in a linear fashion and aligned between the two ferrules 23a and 23b. At this time, circular spaces inside the leaf springs 32a to 32e form a disk-shaped or cylinder-shaped gap. In this manner, the leaf springs 32a to 32e of the spacer 31 function as an integral spacer substantially similar to the spacer 17 of FIGS. 4A and 4B according to Embodiment 1. The rest of the configuration and operation are similar to those of Embodiment 1, and thus the same components are denoted by the same reference symbols to omit the description thereof.

As described above, according to Embodiment 2, which provides the same effect as in Embodiment 1, the tilted leaf springs 32b to 32e shield an optical path of laser light from the connector 20a when the connector 20b is detached from the adapter. Thus, the lens 29a of the connector 20a is configured to be visually unidentifiable directly from the detached connector 20b side, which therefore improves laser safety and can also provide dust resistance with the connector 20b being detached therefrom.

### Embodiment 3

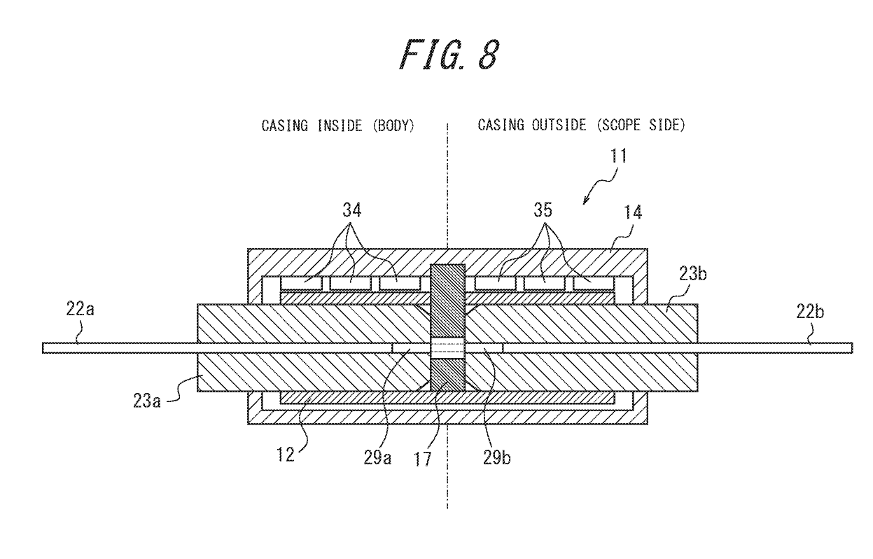

FIG. 8 is a longitudinal section schematically illustrating the connection part of single mode optical fibers, which uses the disclosed adapter according to Embodiment 3. Embodiment 3 includes first photodetectors 34 and second photodetectors 35 between the inner cylindrical part 14 of the adapter housing 11 and the split sleeve 12. The first photodetectors 34 and second photodetectors 35 are optical detection units. The first photodetectors 34 are disposed on the casing outside and the second photodetectors 35 are disposed on the casing inside across the spacer 17. The photodetectors 34, 35 may use, for example, photodiodes (PD). The first photodetectors 34 and the second photodetectors 35 exemplified in FIG. 8 are respectively formed of three photodetectors provided for the colors of RGB. However, the numbers of the first photodetectors 34 and the second photodetectors 35 each may be one, or two or more other than three.

Output signals from the first photodetector 34 and the second photodetector 35 are sent to a detection circuit (not shown) inside the casing and monitored. Light propagating through the single mode optical fiber 22a on the casing inside including a light source partially leaks out from the fiber 22a to pass through the ferrule 23a and the split sleeve 12 and detected by the first photodetectors 34. Accordingly, the first photodetectors 34 can monitor the intensity of light output from the light source. On the other hand, light incident on a clad without being coupled to a core leaks out from the single mode optical fiber 22b on the casing outside to pass through the ferrule 23b and the split sleeve 12 and detected by the second photodetectors 35. Therefore, the output of the second photodetectors 35 depend on the coupling efficiency between the single mode optical fibers 22a and 22b. The rest of the configuration and operation are similar to those of Embodiment 1, and thus the same components are denoted by the same reference symbols to omit the description thereof.

According to Embodiment 5, the first photodetectors 34 and the second photodetectors 35 allow for monitoring of the output of the light source connected to the single mode optical fiber 22a, and also for monitoring the coupling efficiency between the single mode optical fiber 22a on the casing inside and the single mode optical fiber 22b on the casing outside. Accordingly, chronological changes of the light source of the optical system and the connector portion can be detected.

### Embodiment 4

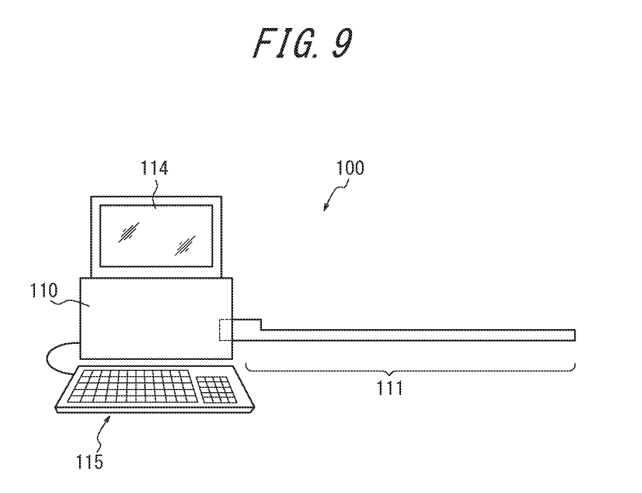

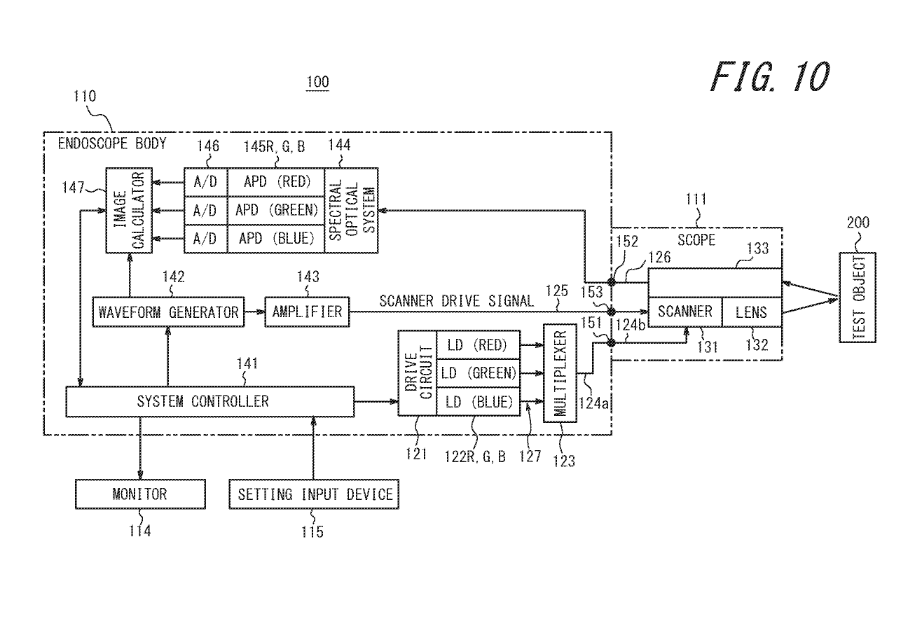

FIG. 9 is a schematic external view of an endoscope apparatus 100 with the disclosed adapter incorporated therein. Further, FIG. 10 is a block diagram illustrating a schematic configuration of the endoscope apparatus 100 of FIG. 9. The endoscope apparatus 100 is configured by including: an endoscope body 110 generally stored in a casing and mounted on a dedicated rack; and a scope 111 detachably connected to the endoscope body 110. The endoscope body 110 serves to control the system as a whole and to generate and process images, and is connected to a dedicated observation monitor 114 and an setting input device 115 for setting observation conditions or the like.

As illustrated in FIG. 10, the endoscope body 110 is configured by including: a system controller 41; a drive circuit 121 electrically connected to the system controller 141; semiconductor lasers (laser diodes (LDs)) 122R, 122G, 122B as semiconductor light sources of red, green, blue; an optical fiber type multiplexer 123; a waveform generator 142; and an amplifier 143. The endoscope body 110 further includes: a spectral optical system 144; avalanche photodiodes (APDs) 145R, 145G, 145B serving as photodetectors; and three analog-to-digital (AD) converters 146 each disposed correspondingly to the respective APDs 145R, 145G, 145B; and an image calculator 147 (image processor).

The LDs 122R, 122G, 122B of the endoscope body 110 each emit laser light as illumination light, which is input to the multiplexer 123 through mutually different single mode fibers 127 and multiplexed, before being output to a single mode optical fiber 124a. The single mode optical fiber 124a is connected to a single mode optical fiber 124 disposed outside the casing, via an optical connection point 151 provided on a side of the housing of the endoscope body 110. The single mode optical fiber 124b passes through inside the scope 111 and extends to the vicinity of the tip thereof. The optical connection point 151 is implemented as the adapter and the connectors described in Embodiments 1 to 3.

The endoscope apparatus 100 is of a scanning type, and includes a scanner 131 at the tip of the scope 111. The scanner 131 is a scanning mechanism for scanning illumination light having passed through the single mode optical fiber 124, relative to an observation site of a test object 200 via the lens 132. For example, the single mode optical fiber 124 connected to a magnet may be oscillatably supported at the tip of the scope 111 and applied with an oscillating magnetic field, to thereby scan the test object 200 with a spiral locus. The use of a piezoelectric element is also known as a method of driving the scanner 131. Further, various scanning loci may be adopted including raster scanning and Lissajous scanning, without being limited to the spiral locus.

The waveform generator 142 of the endoscope body 110 generates a drive signal, which is amplified by the amplifier 143 to pass through, via an electrical connection point 153 between the endoscope body 110 and the scope 111, a scanner drive signal line 125 extending through inside the scope 111, to be supplied to the scanner 131. In this manner, the scanner 131 is controlled by the system controller 141 connected to the waveform generator 142 of the endoscope body 110.

The test object 200 irradiated with illumination light provides light (detecting light) such as reflected light, scattered light, and fluorescence, which is partially incident on the detection fiber bundle 126 from a detection fiber bundle incident end 133. The detection fiber bundle incident end 133 may be arranged, for example, along the outer periphery of the leading end of the scope 111 facing the test object 200 while having the incident surface facing toward the test object 200, or may be disposed as bundled in part of the tip of the scope 111. The detection fiber bundle 126 is optically connected to the detection fiber bundle on the endoscope body 110 side, at an optical connection point 152 between the endoscope body 110 and the scope 111.

The detecting light propagated to the endoscope body 110 is separated into red, green, and blue components by the spectral optical system 144, which are each detected by the APDs 145R, 145G, 145B. The spectral optical system 144 may be formed by a publicly-known method using dichroic mirrors, diffractive elements, and color filters. Detecting lights of red, green, and blue are subjected to photoelectric conversion in the APD 145R, 145G, 145B to be converted into pixel signals, which are then converted into digitals signals by the A/D converter 146 and sent to the image calculator 147.

The image calculator 147 is controlled by the system controller 141 synchronously with the waveform generator 142, and associates digital pixel signals of red, green, and blue which are sequentially sent, with the scanning position of illumination light scanned by the scanner 131, to thereby define the pixel position of the pixel signal chronologically obtained. In this manner, pixel signals per one frame are sequentially generated as two-dimensional image data. The two-dimensional data thus generated is sent to and displayed on the monitor 114 while stored in a storage device (not shown).

As described above, the adapter and the connector according to any of Embodiments 1 to 3 are applied to the optical connection point 151 of the single mode optical fiber between the inside of the endoscope body 110 and the scope 111 outside. In the endoscope apparatus 100, the scope 111 is detached, after each use, from the endoscope body 110 for cleaning purposes, and the connection efficiency may vary along with the attachment/removal of the connector. The adapter according to any of Embodiments 1 to 3 reduces such variations in connection efficiency, to thereby obtain stable connection efficiency.

The present disclosure is not limited to Embodiments above, and may be subjected to a number of modifications and alterations. For example, the application of the disclosed connector is not limited to an endoscope, and the connector may be used for connecting communication optical fibers and in a scanning microscope with laser light as the light source. In Embodiments disclosed herein, FC connectors and adapters as the standards in the optical communication field are illustrated as exemplary applications of the disclosure, but other standards such as SC type, ST type, MU type, and LC type are also applicable, not to mention proprietary standards having the same function. The type of the connector to be paired does not necessarily need to be the same, and different type of connectors is also applicable to be paired.

### REFERENCE SIGNS LIST

10 adapter
11 adapter housing
12 split sleeve
12a slit
13a, 13b outer cylindrical part (connector connection part)
14 inner cylindrical part (connector connection part)
15a, 15b external thread (connector connection part)
16a, 16b key receiver
17 spacer
17a space determiner
17b angle determiner
17c gap
18 spacer holder
19 drilled hole
20a, 20b connector
21a, 21b connector housing
22a, 22b single mode optical fiber
23a, 23b ferrule
24a, 24b cylindrical part
25a, 25b key
26a, 26b coupling nut
27a, 27b spring
28a, 28b step
29a, 29b lens
30a, 30b magnet
31 spacer
32a to 32e leaf spring
33a to 33e gap
34 first photodetector (optical detection unit)
35 second photodetector (optical detection unit)
100 endoscope apparatus
110 endoscope body
111 scope
114 monitor
115 setting input device
121 drive circuit
122R, 22G, 22B LD (semiconductor light source)
123 multiplexer
124 single mode fiber
125 scanner drive signal line
126 detection fiber bundle
127 single mode fiber
131 scanner
132 lens
133 detection fiber bundle incident end
141 system controller
142 waveform generator
143 amplifier
144 spectral optical system
145R, 145G, 145B APD (photodetector)
146 A/D converter
147 image calculator (image processor)
151 optical connection point (single mode optical fiber connection point)
152 optical connection point (multimode optical fiber connection point)
153 electrical connection point
200 test object

1. A connection adapter connecting connectors to each other, the connectors each having a ferrule incorporating therein a leading end of a single mode optical fiber, the connection adapter comprising:
an adapter housing having two connector connection parts opposing to each other;
a split sleeve disposed between the two connector connection parts; and
a spacer having a space determiner disposed in the split sleeve and an angle determiner that passes through the slit of the split sleeve to be secured relative to the adapter housing,
wherein the two connector connection parts are each connected to each of the connectors, causing the ferrules of the connectors to be fitted into the split sleeve and secured across the spacer, so that the single mode optical fibers of the connectors are optically connected to each other via no point of contact therebetween.

2. The adapter according to claim 1, wherein the angle determiner is configured to be capable of adjusting angles around the central axis of the split sleeve with respect to the adapter housing.

3. The adapter according to claim 1, wherein the space determiner includes a plurality of plate-like members, and when one of the connector connection parts is not connected to the connector, at least some of the plurality of plate-like members are tilted at different angles toward the connector connection part side not connected to the connector, so as to shield an optical path of light emitted from the single mode optical fiber of the connector connected to the other connector connection part.

4. The adapter according to any one of claims 1, further comprising an optical detection unit between the adapter housing and the split sleeve.

5. The adapter according to claim 4, wherein the optical detection unit is disposed on each side of the two connector connection parts of the spacer.

6. An endoscope apparatus, comprising:
a casing which accommodates therein a laser light source or is connected to a laser light source;
a scope which irradiates an object with laser light output from the casing and receives signal light obtained from the object;
an image processor which generates an image, based on the signal light received by the scope; and
a connection adapter which connects, between the casing and the scope, connectors each having a ferrule which incorporates therein a leading end of an optical fiber,
wherein the connection adapter includes:
an adapter housing having two connector connection parts opposing to each other;
a split sleeve disposed between the two connection parts; and
a spacer having a space determiner disposed in the split sleeve and an angle determiner that passes through the slit of the split sleeve to be secured to the adapter housing,
wherein the two connector connection parts are each connected to each of the connectors, causing the ferrules of the connectors to be fitted into the split sleeve and secured across the spacer, so that the single mode optical fibers of the connectors are optically connected to each other via no point of contact therebetween.
